(19) United States
(12) Patent Application Publication
(10) Pub. No.: US 2025/0128000 A1
(43) Pub. Date: Apr. 24, 2025

(54) INSUFFLATOR SAFETY VALVE HAVING ELECTRICALLY VARIABLE CRACKING PRESSURE

(71) Applicant: Conmed Corporation, Largo, FL (US)

(72) Inventor: Michael L. Koltz, Aurora, CO (US)

(73) Assignee: Conmed Corporation, Largo, FL (US)

(21) Appl. No.: 18/684,914

(22) PCT Filed: Aug. 23, 2022
(86) PCT No.: PCT/US2022/041170

§ 371 Date: Feb. 20, 2024

Related U.S. Application Data
(60) Provisional application No. 63/246,018, filed on Sep. 20, 2021.

(51) Int. Cl. A61M 13/00; A61M 39/24

(52) U.S. Cl. CPC A61M 13/003; A61M 39/24; A61M 2039/242; A61M 2202/0007; A61M 2202/02; A61M 2205/103; A61M 2205/3327; A61M 2205/3334; A61M 2205/3344; A61M 2205/50

(57) ABSTRACT

An insufflator is disclosed for delivering surgical gas from a gas source to a patient's body cavity during a surgical procedure, which includes an electrically variable safety valve adapted and configured to prevent over-pressurization of the patient's body cavity during the surgical procedure by venting the surgical gas in the patient's body cavity to atmosphere when a cracking pressure of the variable safety valve is exceeded.

### CROSS-REFERENCE TO RELATED APPLICATIONS

The subject application claims the benefit of priority to U.S. Provisional Patent Application Ser. No. 63/246,018 filed Sep. 20, 2021, the disclosure of which is incorporated herein by reference in its entirety.

### BACKGROUND OF THE DISCLOSURE

### 1. Field of the Disclosure

The subject application is directed to endoscopic surgery, and more particularly, to an insufflator for delivering surgical gas to a patient's body cavity, which includes a safety valve having electrically variable cracking pressure.

### 2. Description of Related Art

Laparoscopic or “minimally invasive” surgical techniques are becoming commonplace in the performance of procedures such as cholecystectomies, appendectomies, hernia repair and nephrectomies. These procedures commonly involve filling or “insufflating” the abdominal cavity with a pressurized fluid, such as carbon dioxide, to create an operating space, which is referred to as a pneumoperitoneum.

Insufflation associated with laparoscopic surgery presents an inherent patient risk associated with over-pressurization of the abdominal cavity. Over-pressurization has led to numerous patient injuries in both adult and pediatric victims. Most insufflators in use today incorporate a solely mechanical safety valve plumbed into the intermediate pressure node of the gas insufflation network. The safety valve serves as a final means of protection against mechanical, electrical, and software failures which could result in pressure exceeding a pneumoperitoneal control set-point.

While insufflator safety valves known in the prior art are adjustable, they are typically fixed to a specified value or cracking pressure only at the time of manufacture or subsequent service. This non-user-adjustable nature of the valve requires it to be set above maximum normal expected pressure levels to prevent the valve from intermittently leaking during surgical use. These leak preventative pressure set-points are commonly above the threshold for mitigating over-pressurization in most applications when used on a majority of the patient population.

It would be beneficial to provide an insufflator safety valve wherein the cracking pressure or set-point of the valve that can be adjusted intra-operatively. This adjustability would enable cracking pressures to be set much lower than safety valves described in the prior art, without frequent valve opening. Furthermore, the cracking pressure could be lowered for surgeries requiring less gas flow or for patients with smaller peritoneal volume, such as children.

### SUMMARY OF THE DISCLOSURE

The subject disclosure is directed to a new and useful insufflator for delivering surgical gas from a gas source to a patient's body cavity during a surgical procedure. The insufflator includes an electrically variable safety valve that is adapted and configured to prevent over-pressurization of the patient's body cavity during the surgical procedure by venting the surgical gas in the patient's body cavity to atmosphere when a cracking pressure of the variable safety valve is exceeded. In use, when a pressure differential across the valve exceeds the cracking pressure, the valve is “cracked open” and gas can flow.

In accordance with the subject disclosure, the variable safety valve includes an electric motor for intra-operatively adjusting the cracking pressure of the variable safety valve, a motor controller for commanding the electric motor to adjust the cracking pressure of the variable safety valve based upon a feedback signal. In one embodiment of the subject disclosure, the feedback signal is provided by an input signal from an operator. In another embodiment, the feedback signal is provided by a signal from a sensor.

When the input signal is provided by an operator, the motor controller will command the electric motor to adjust the cracking pressure of the variable safety valve based on an operator selected cracking pressure selected from a set of at least two discrete cracking pressure set points. When the signal is provided by a sensor, the motor controller commands the electric motor to adjust cracking pressure of the variable safety valve based upon a feedback signal from a flow sensor and/or a pressure sensor.

The insufflator further includes a flow sensor for monitoring a flow rate of the surgical gas delivered to the patient's body cavity, a pressure sensor for monitoring the pressure of the surgical gas delivered to the patient's body cavity, a control valve for controlling the delivery of surgical gas from the insufflator to the patient's body cavity, and a pressure regulator for controlling the pressure of surgical gas delivered to the insufflator from the gas source.

In accordance with the subject disclosure, the electric motor is a stepper motor that drives a lead nut which translates rotary motion into linear motion by way of a lead screw. The lead screw drives a pressure plate in contact with one end of a valve spring, effectively changing an installed length of the valve spring. The shortening of the installed length of the valve spring linearly correlates with increasing the cracking pressure of the variable safety valve, which permits gas flow when the gas pressure acting against a front face of the diaphragm exceeds a spring force applied to a back side of the diaphragm.

These and other features of the electrically variable insufflator safety valve of the subject disclosure will become more readily apparent from the following detailed description of the preferred embodiments taken in conjunction with the drawings.

### BRIEF DESCRIPTION OF THE DRAWINGS

So that those skilled in the art will readily understand how to make and use the insufflator safety valve of the subject disclosure without undue experimentation, embodiments thereof will be described in detail herein below with reference to the figures wherein:

FIG. 1 is a schematic representation of an insufflator constructed in accordance with subject disclosure; and

FIG. 2 is a cross-sectional view of the electrically variable safety valve of the subject disclosure, which is incorporated into the insufflator shown in FIG. 1.

### DESCRIPTION OF THE DISCLOSED EMBODIMENTS

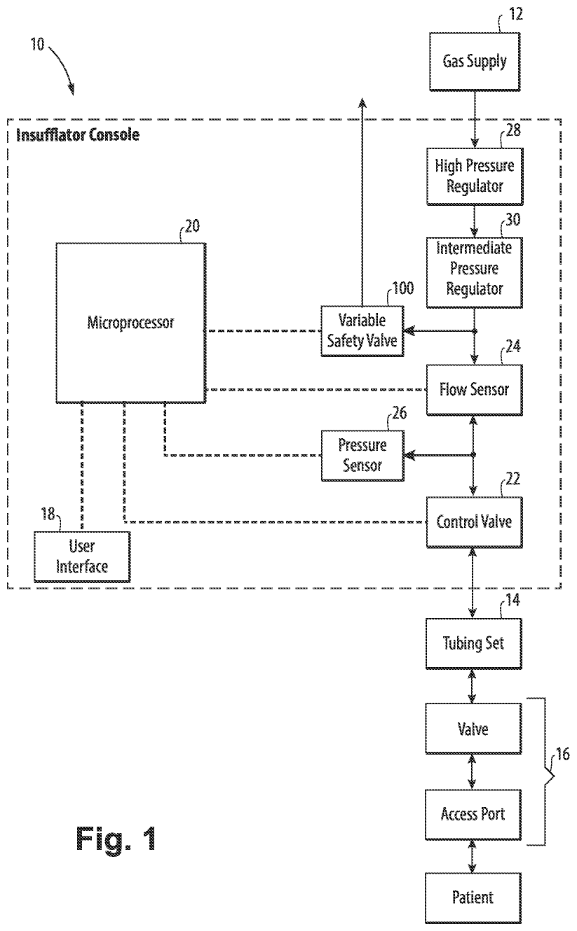

Referring now to the drawings wherein like reference numeral identify similar features or components of the subject disclosure, there is illustrated in FIG. 1 a schematic representation of an insufflator console 10 incorporating the variable safety valve 100 of the subject disclosure. The insufflator console 10 receives pressurized surgical gas from a gas supply source 12 and delivers pressure regulated surgical gas to a patient's body cavity by way of a tube set 14 that communicates with a valve sealed access port or trocar 16.

The insufflator 10 includes a graphical user interface 18 or GUI and a microprocessor or motor controller 20 for controlling the variable safety valve 100 and a separate flow control valve 22 based on user inputs through the GUI 18 and feedback signals from a flow sensor 24 and/or a pressure sensor 26.

In use, surgical gas entering the insufflator console 10 from gas supply 12 passes initially through a high pressure regulator 28 and then through an intermediate pressure regulator 30. The gas flow then interacts with the flow sensor 24 and pressure sensor 26 before flowing to the control valve 22, which moderates or otherwise controls the delivery of gas from the insufflator console 10 to the tube set 14 and attached to the valve sealed access port 16.

The variable safety valve 100 of the subject disclosure is positioned downstream from the intermediate pressure regulator 30 and upstream from the flow sensor 24. It is adapted and configured to direct or otherwise vent gas from the patient's abdominal cavity, out of the insufflator console 10 and into the surrounding atmosphere when it is actuated, so as to advantageously prevent over-pressurization of the patient's abdominal cavity. As explained in more detail below, the variable safety valve 100 is actuated when a pressure differential across the valve exceeds the valves' cracking pressure or pneumoperitoneal control set-point.

It is envisioned that the variable safety valve 100 of the subject disclosure could be incorporated into the low pressure insufflation manifold of the multi-modal gas delivery system disclosed in commonly assigned U.S. Patent Application Publication 2022/0233791, the disclosure of which is incorporated herein by reference in its entirety.

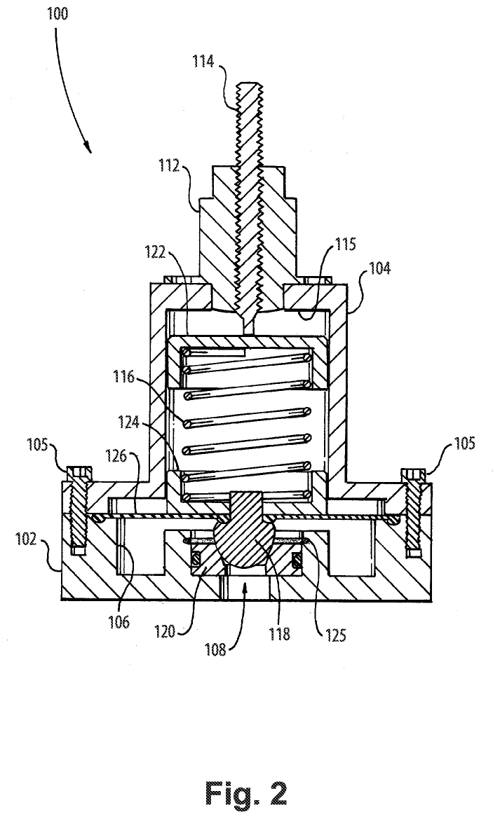

Referring now to FIG. 2, the variable safety valve includes 100 a base 102 and a housing 104. The housing is connected to the base 102 by threaded fasteners 105. The base 102 of valve 100 has an interior pressure cavity 106 that communicates with the gas delivery flow path of insufflator 10, downstream from the intermediate pressure regulator 30 and upstream from the flow sensor 24, as illustrated in FIG. 1. The base 102 also has a vent port 108, which communicates with the pressure cavity 106 and with the surrounding atmosphere.

The flow control element of safety valve 100 is a poppet valve member 118, which is associated with a valve seat 120 located between the pressure cavity 106 and the vent port 108 and secured in place by a lock ring 125. The poppet valve member 118 is operatively associated with a flexible diaphragm 126 that extends across the interior pressure cavity 106 and is securely fixed about its outer periphery between the base 102 and valve housing 104. The front face of the diaphragm 126 defines an upper boundary of the pressure cavity 106 and it will react to pressure changes therein.

The rear or back face of the diaphragm 126 is operatively associated with a coiled valve spring 116 supported with the interior chamber 115 of housing 104 of valve 100. The coiled valve spring 116 is sandwiched between an upper pressure plate 122 and a lower pressure plate 124. The lower pressure plate 124 is operatively associated with the back face of diaphragm 126 while the upper pressure plate 122 is operatively connected to an electric motor (e.g., a stepper motor) 112. The upper pressure plate 122 is connected to the electric motor 112 by way of a torque/force multiplying drive mechanism in the form of a lead screw 114.

Those skilled in the art will readily appreciate that the motor 112 need not be a stepper motor, in that any electric motor with position feedback can be substituted. The stepper motor is desirable however, as position can be controlled by step inputs eliminating the need for a position feedback loop. Those skilled in the art will also appreciate that the drive mechanism need not be direct or translate to linear motion. Alternatives incorporating gears, rotary actuation, and non-linear springs are also envisioned and well within the scope of the subject disclosure.

In use, when the cracking pressure of the valve 100 is adjusted intra-operatively, the stepper motor 112 drives a lead nut (not shown) which translates rotary motion into linear motion by way of the lead screw 114. The lead screw motion drives the upper pressure plate 122 in contact with the upper end of valve spring 116, which effectively changes the installed length of the valve spring 116 within valve housing 104. Shortening the installed length of the valve spring 116 linearly correlates with increasing the cracking pressure of the safety valve 100, which permits gas flow when the gas pressure acting within pressure chamber 106 against the front face of the diaphragm 126 exceeds the spring force applied to the back side of the diaphragm 126 by the valve spring 116.

In one embodiment of the subject disclosure, a user or operator can select a cracking pressure for the valve 100 from at least two discrete cracking pressure set points by way of the graphical user interface 18 of the insufflator 10. In another embodiment of the subject disclosure, the operator can continuously chose from a group of selectable set points throughout a cracking pressure range. In yet another embodiment of the subject disclosure, the cracking pressure of the valve 100 is continuously and automatically set by the processor/motor controller 20 based on a feedback signal from the flow sensor 24 and/or the pressure sensor 26. This automated control facilitated by processor 20 can be implemented by way of appropriate electrical circuitry with or without associated software.

The subject disclosure eliminates the user-based set-point decision process and enables the insufflation system to actively adapt to downstream configurations (e.g., tubes, valves, access ports, etc.) and patient variation by monitoring pressure, flow, or a combination thereof. For example, the insufflation system can set the initial cracking pressure to a low cracking pressure of 5 mmHg to maintain a high level of over-pressurization safety and automatically increase the cracking pressure set-point as the measured gas delivery rate at flow sensor 24 increases or the measured intermediate node pressure increases at pressure sensor 26, while never permitting the set-point to exceed the maximum 80 mmHg level or a lower user selected/configurable value.

Under this scenario, if a fault occurs leading to an over-pressurization hazard, the set point or cracking pressure would never be above the 80 mmHg threshold, and when considered as a fluctuating value over time, the cracking pressure at the time of the fault would likely be closer to the pneumoperitoneal set point rather than the 80 mmHg upper limit.

While the subject disclosure has been shown and described with reference to preferred embodiments, those skilled in the art will readily appreciate that changes and/or modifications may be made thereto without departing from the scope of the subject disclosure.

What is claimed is:

1. An insufflator for delivering surgical gas from a gas source to a patient's body cavity during a surgical procedure, comprising:
an electrically variable safety valve adapted and configured to prevent over-pressurization of the patient's body cavity during the surgical procedure by venting the surgical gas in the patient's body cavity to atmosphere when a cracking pressure of the variable safety valve is exceeded.

2. An insufflator as recited in claim 1, wherein the variable safety valve includes an electric motor for intra-operatively adjusting the cracking pressure of the variable safety valve.

3. An insufflator as recited in claim 2, further comprising a motor controller for commanding the electric motor to adjust the cracking pressure of the variable safety valve based upon a feedback signal.

4. An insufflator as recited in claim 3, wherein the feedback signal is provided by an input signal from an operator.

5. An insufflator as recited in claim 3, wherein the feedback signal is provided by a signal from a sensor.

6. An insufflator as recited in claim 4, wherein the motor controller commands the electric motor to adjust the cracking pressure of the variable safety valve based on an operator selected cracking pressure selected from a set of at least two discrete cracking pressure set points.

7. An insufflator as recited in claim 5, wherein the motor controller commands the electric motor to adjust cracking pressure of the variable safety valve based upon a feedback signal from a flow sensor and/or a pressure sensor.

8. An insufflator as recited in claim 7, further comprising a flow sensor for monitoring a flow rate of the surgical gas delivered to the patient's body cavity.

9. An insufflator as recited in claim 7, further comprising a pressure sensor for monitoring the pressure of the surgical gas delivered to the patient's body cavity.

10. An insufflator as recited in claim 1, further comprising a control valve for controlling the delivery of surgical gas from the insufflator to the patient's body cavity.

11. An insufflator as recited in claim 1, further comprising a pressure regulator for controlling the pressure of surgical gas delivered to the insufflator from the gas source.

12. An insufflator as recited in claim 2, wherein the electric motor is a stepper motor that translates rotary motion into linear motion by way of a lead screw.

13. An insufflator as recited in claim 12, wherein the lead screw drives a pressure plate in contact with one end of a valve spring, effectively changing an installed length of the valve spring.

14. An insufflator as recited in claim 13, wherein shortening the installed length of the valve spring linearly correlates with increasing the cracking pressure of the variable safety valve, which permits gas flow when the gas pressure acting against a front face of the diaphragm exceeds a spring force applied to a back side of the diaphragm.

15. An insufflator for delivering surgical gas from a gas source to a patient's body cavity during a surgical procedure, comprising:
a) a variable safety valve adapted and configured to prevent over-pressurization of the patient's body cavity during the surgical procedure by venting the surgical gas in the patient's body cavity to atmosphere when a cracking pressure of the variable safety valve is exceeded;
b) an electric motor for intra-operatively adjusting the cracking pressure of the variable safety valve; and
c) a motor controller for commanding the electric motor to adjust the cracking pressure of the variable safety valve based upon an operator selected cracking pressure.

16. An insufflator as recited in claim 15, wherein the operator selected cracking pressure is selected from a set of at least two discrete cracking pressure set points.

17. An insufflator for delivering surgical gas from a gas source to a patient's body cavity during a surgical procedure, comprising:
a) a variable safety valve adapted and configured to prevent over-pressurization of the patient's body cavity during the surgical procedure by venting the surgical gas in the patient's body cavity to atmosphere when a cracking pressure of the variable safety valve is exceeded;
b) an electric motor for intra-operatively adjusting the cracking pressure of the variable safety valve; and
c) a motor controller for commanding the electric motor to adjust the cracking pressure of the variable safety valve based upon a feedback signal from a sensor.

18. An insufflator as recited in claim 17, wherein the feedback signal is generated by a flow sensor and/or a pressure sensor.

19. An insufflator as recited in claim 18, further comprising a flow sensor for monitoring a flow rate of the surgical gas delivered to the patient's body cavity.

20. An insufflator as recited in claim 18, further comprising a pressure sensor for monitoring the pressure of the surgical gas delivered to the patient's body cavity.
